(19) United States
(12) Patent Application Publication
(10) Pub. No.: US 2018/0169437 A1
(43) Pub. Date: Jun. 21, 2018

(54) RESULT-DRIVEN RADIATION THERAPY TREATMENT PLANNING

(71) Applicant: SIRIS MEDICAL, INC., Redwood City, CA (US)

(72) Inventors: Colin Morehouse CARPENTER, San Carlos, CA (US); Adam J. PATTISON, San Mateo, CA (US)

(21) Appl. No.: 15/897,975

(22) Filed: Feb. 15, 2018

Related U.S. Application Data
(63) Continuation-in-part of application No. 15/752,236.
(60) Provisional application No. 62/204,470, filed on Aug. 13, 2015.

(51) Int. Cl. A61N 5/10; G16H 20/40; G16H 10/60; G16H 30/40

(52) U.S. Cl. CPC A61N 5/1038; A61N 5/1039; G16H 30/40; G16H 10/60; G16H 20/40

(57) ABSTRACT

The treatment planning engine empowers radiation treatment decision makers, such as a clinician, to efficiently identify optimal radiation treatment results for a given patient. Specifically, the treatment planning engine may identify prior patients associated with previously-administered treatment plans and treatment results, wherein the identified prior patients are similar to a current patient who is to receive radiation treatment. The treatment planning engine generates a set of treatment results for the current patient treatment based on the identified prior patient treatment results. A user interface displays the generated set of treatment results. The clinician is able to input specific criteria, such as features of the patient's medical history, into the user interface to filter and modify the displayed set of treatment results based on a similarity between the medical history of the prior patients and the current patient.

### CROSS REFERENCE TO RELATED APPLICATIONS

This application is a continuation-in-part of U.S. application Ser. No. 15/752,236, filed Feb. 12, 2018, which is a national phase application of International Application No. PCT/US2016/047041, with an international filing date of Aug. 15, 2016, which claims the benefit of U.S. Provisional Application No. 62/204,470, filed Aug. 13, 2015, each of which is incorporated by reference in its entirety.

### BACKGROUND

This invention relates generally to radiation treatment planning, and more specifically to planning relating to toxicity outcomes or treatment efficacy.

The process of determining the optimal therapy for patients afflicted with cancer has become an increasingly complex task, due to the overwhelming degrees of freedom and constraint priorities, which often change depending on the clinical care team, attending physicians, physicists, and available technology, and the uniqueness of the disease. Prospective clinical trials tend to have limited outcome scope and specificity, and homogenize a population. Complicating this is the variability in therapy such as radiation therapy, where the dosimetrist who creates a radiation plan has a difficult challenge in creating the optimal plan; this process is further challenged because the physicians and physicists are sporadically connected to the treatment planning process due to of other demands on their time. In addition, the clinical care team does not know the precise probabilities of adverse events (including likely toxicity to anatomical structures and treatment efficacy) resulting from treatment during the treatment planning process, as they instead rely on data from studies with homogenized patient populations. Thus, the members of the clinical care team are limited in their abilities to make informed decisions about certain tactical trade-offs in a patient's treatment.

### SUMMARY

A treatment planning engine informs a patient's clinical care team of the probabilities of adverse events as a result of treatment for the patient during the process of planning the patient's treatment. By providing real-time information as to the likely therapeutic response of the disease, as to the toxicity of the treatment to anatomical structures of the patient, and as to the efficacy of the treatment for the patient, the engine allows the clinical care team to make informed decisions about tactical trade-offs in the patient's treatment. The treatment planning engine described herein empowers radiation treatment decision makers, such as a clinician, to efficiently identify an optimal radiation treatment result for a given patient.

In operation, the treatment planning engine imports data for a given patient, such as patient images and patient contours that may have been previously defined by a clinician or a medical history of the given patient. The treatment planning engine generates a set of treatment results (e.g., results that include a particular toxicity to anatomical structure near to the tumor, a specific efficacy of the treatment, including probability of tumor recurrence, period of time until recurrence, etc.). The generated set of treatment results are comprised of treatment result matches, which are matches to results of dose plans that were previously administered to other patients, where these matched results are ones that may be recommended as effective for the given patient as well. The treatment planning engine presents the generated set of treatment results to the clinician for evaluation.

A user interface displays the generated set of treatment results as recommended treatment results for the patient. The clinician is able to input specific criteria, such as features of the patient's medical history, into the user interface to filter and modify the displayed treatment results based on a similarity between the medical history of the current patient and medical histories of the prior patients who had the previously-administered dose plans.

### BRIEF DESCRIPTION OF THE DRAWINGS

FIG. 1 illustrates a block diagram of a system environment for radiation treatment planning and delivery, according to an embodiment.

FIG. 2 illustrates a block diagram of a system for determining the recommended dose plans that are output by the dose prediction module, according to an embodiment.

FIG. 3 illustrates a system environment for generating a radiation dose plan prediction model, according to an embodiment.

FIG. 4 illustrates a block diagram of a system for generating a set of treatment results that are output by the treatment results module, according to an embodiment.

FIG. 5 illustrates a system environment for generating a treatment results prediction model, according to an embodiment.

FIG. 6 illustrates a system for optimizing a selected treatment result, according to an embodiment.

FIG. 7 illustrates an exemplary user interface for visualizing treatment results, according to an embodiment.

FIG. 8 illustrates an exemplary user interface for visualizing treatment results, according to an additional embodiment.

FIG. 9 illustrates a flow diagram illustrating the steps for clinician-directed radiation treatment planning, according to an embodiment.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### System Architecture

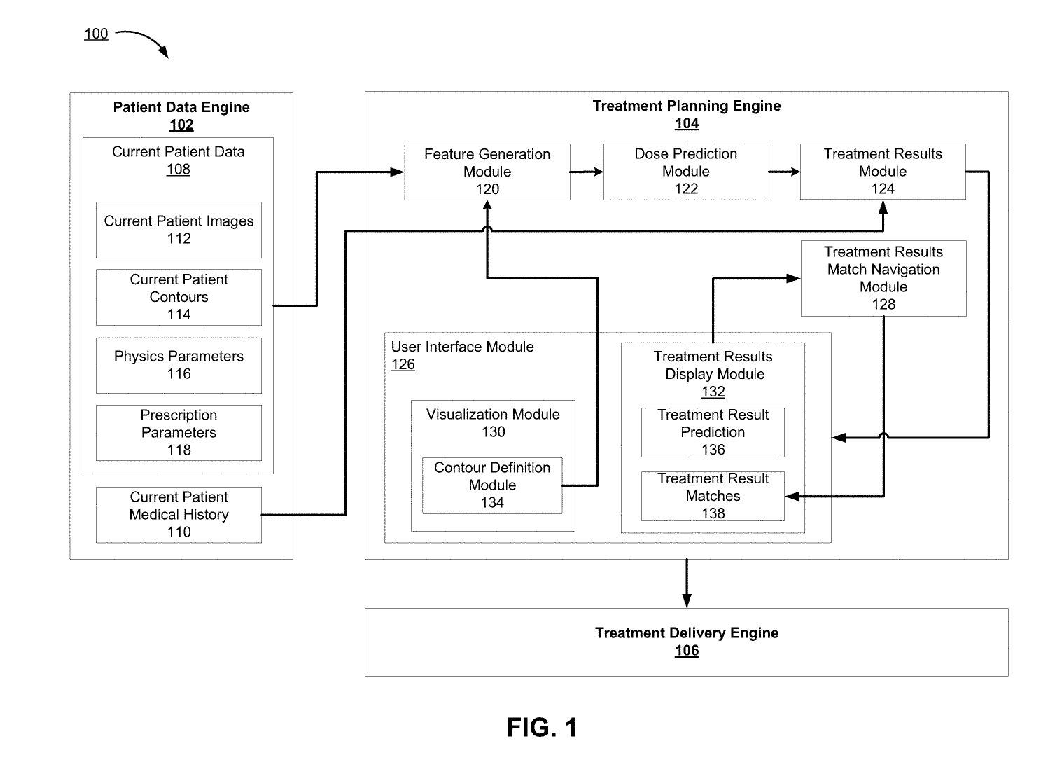

FIG. 1 illustrates a block diagram of a system environment 100 for radiation treatment planning and delivery, according to an embodiment. Some embodiments of the system environment 100 have different modules than those described here. Similarly, the functions can be distributed among the modules in a different manner than is described here. In certain embodiments of the system 100, a “treatment result” may be referred to as a “treatment outcome.” Both phrases indicate the results of delivering a radiation therapy treatment plan (i.e., a “dose plan”) to a patient, in terms of the treatment plan's toxicity to organs near the tumor, the plan's efficacy for treatment of the tumor, etc. As shown, the system environment 100 includes a patient data engine 102, a treatment planning engine 104, and a treatment delivery engine 106.

The patient data engine 102 provides data associated with patients needing radiation treatment. The patient data may be imported into the patient data engine 102. In the embodiment of FIG. 1, data associated with a current patient includes current patient data 108 and current patient medical history 110.

The current patient data 108 is data associated with a current patient that is used to generate features associated with the current patient. The patient data engine 102 transmits the current patient data 108 to the treatment planning engine 104 to generate the features associated with the current patient. These generated features embody the relationship between radiation delivery and dose. These generated features can be compared to similarly generated features of prior patients for the purpose of identifying one or more prior patients that are the most similar to the current patient. By matching these generated features, prior treatment results may be drawn from a database of previously-administered treatment plans to find a prior patient's treatment plan (i.e., dose plan) that is the closest match to providing an optimal treatment result for the current patient. The prior patient's treatment plan may inform the treatment planner or clinician of what may be expected when a similar treatment is delivered to the current patient. The generated features may also be used in prediction models to create dose predictions and treatment result predictions for a patient. In the embodiment of FIG. 1, current patient data 108 includes current patient images 112, current patient contours 114, physics parameters 116, and prescription parameters 118.

The current patient images 112 may include visual representations of the interior of a patient's body for medical purposes. These current patient images 112 may be produced by a medical imaging technology, such as computed tomography (CT), magnetic resonance imaging (MRI), X-ray, fluoroscopy, ultrasound, nuclear medicine including positron emission tomography (PET), or any other suitable medical imaging technology. In the embodiment of FIG. 1, the current patient images 112 may be a plurality of images, wherein each image illustrates the same perpendicular cross-section of the interior of the patient's body but at various depths of the patient's body. The current patient images 112 may have associated patient image parameters, such as distance, volume, geometric relationship, importance of structures and surrounding structures, and/or the like. The current patient images 112 and the associated patient image parameters may be imported into the patient data engine 102.

The current patient contours 114 are a set of contours identifying the three-dimensional tumor volumes and the anatomical structures located in the surrounding region of the tumor volumes that are captured in the current patient images 112. The contours identifying the three-dimensional tumor volumes indicate the targeted area of radiation dose delivery, while the contours identifying the surrounding anatomical structures indicate structures that may be at risk from the radiation dose delivery. Prior to importing the current patient data 108 into the patient data engine 102, the current patient contours 114 may be defined in each current patient image 112 by a clinician. In some embodiments, the current patient contours 114 may be automatically generated for the current patient images 112 using contouring techniques known in the field. The current patient contours 114 may be automatically generated using historical contours created for the current patient or a different patient having similar imaging data.

The physics parameters 116 are parameters of radiation delivery. Physics parameters may include penumbra, aperture, incident angle, beam energy, radiation type, depth of structure, existence of bolus, resolution of collimator, dose delivery rate, type of Treatment Planning System used, and other radiation delivery descriptive data. In some embodiments, the physics parameters 116 may not be included as inputs into the treatment planning engine 104.

The prescription parameters 118 are parameters regarding the method of radiation delivery. Prescription parameters may include fractionation schedule, treatment margin, number of beams or arcs, interpretation of contours, the clinicians involved in the treatment planning, and/or the like. In some embodiments, the prescription parameters 118 may not be included as inputs into the treatment planning engine 104.

In some embodiments, the current patient data 108 may further include disease parameters for the patient, such as disease stage, prior or post treatment therapy, prior radiation therapy, prior radiation damage to nearby tissue, disease type, disease histology, extent of the disease, prior disease, and/or the like.

The current patient medical history 110 is the medical history of a current patient that is used to improve the precision of the generated set of treatment results. The current patient medical history 110 may include available therapeutic options, especially considering ability to pay for available therapies, genetic results, digital genetics, in vitro lab results, prior treatment history, comorbidities, etc. Examples of comorbidities include, but are not limited to, smoking history, cardiac or lung function, kidney function, diabetes, etc. Knowledge of a patient's medical history better informs the clinician of expected treatment results. For example, a patient who is a smoker is expected to have worse treatment results for lung function compared to a similar patient who is a non-smoker.

The treatment planning engine 104 processes patient data associated with a patient and recommends different treatment results (i.e., the generated set of treatment results) to the patient's clinician, thus enabling the clinician to efficiently identify the optimal treatment result for the patient. Some embodiments of the treatment planning engine 104 have different modules than those described here. Similarly, the functions can be distributed among the modules in a different manner than is described here. The treatment planning engine 104 includes a feature generation module 120, a dose prediction module 122, a treatment results module 124, a user interface module 126, and a treatment result match navigation module 128.

The feature generation module 120 generates features associated with the current patient. In the embodiment of FIG. 1, the feature generation module 120 imports the current patient data 108 which includes current patient images 112, current patient contours 114, physics parameters 116, and prescription parameters 118. The feature generation module 120 uses the current patient data 108 to generate features associated with the current patient that can be compared to similar generated features associated with prior patients. These generated features can be used to identify, from a database, one or more prior patients that are most similar to the current patient. As previously described, by matching these generated features, prior treatment results may be drawn from a database of previously administered treatment plans to find a prior patient's treatment plan that is the closest match to providing an optimal treatment result for the current patient. The prior patient's treatment plan may inform the treatment planner or clinician of what may be expected when a similar treatment is delivered to the current patient.

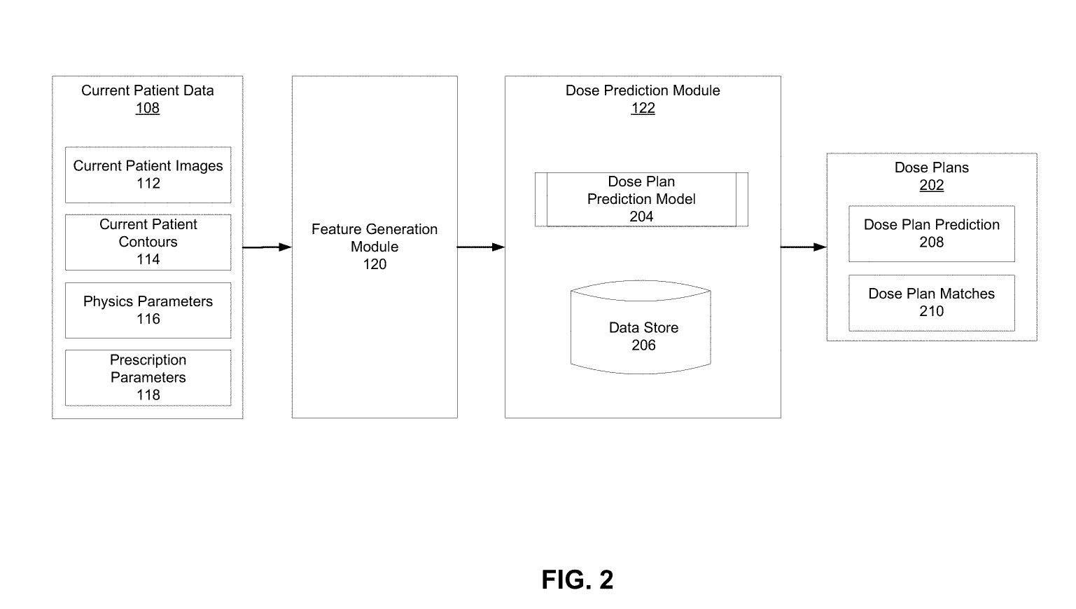

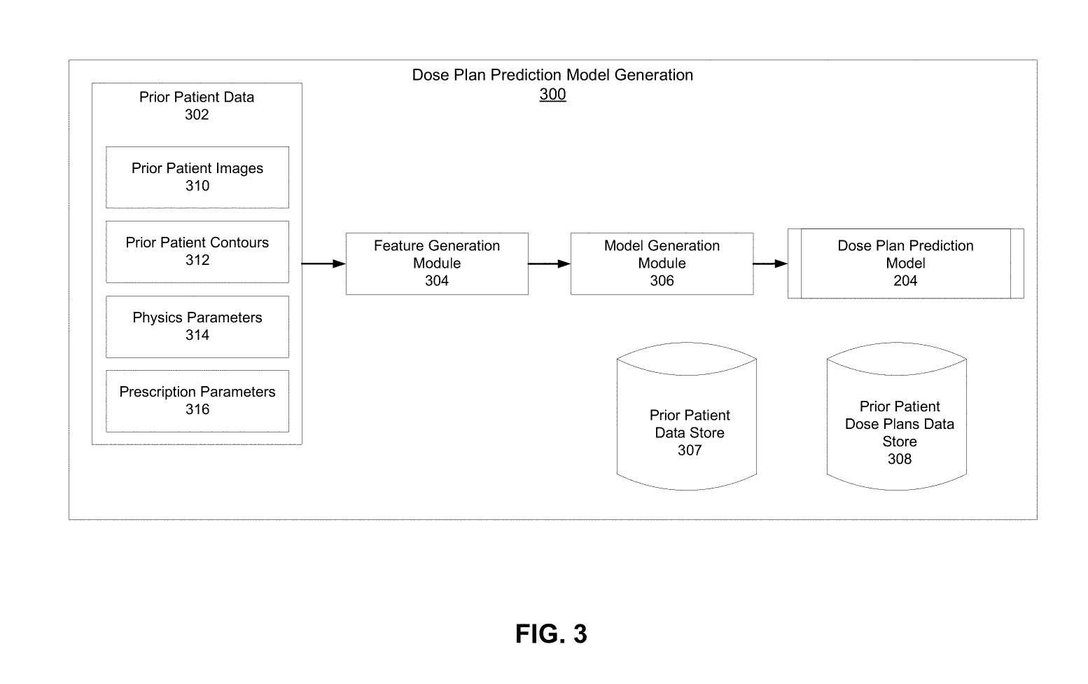

The dose prediction module 122 predicts the radiation dose delivered to tumor volumes and surrounding anatomical structures. In the embodiment of FIG. 1, the dose prediction module 122 outputs a set of recommended dose plans, which includes a plurality of dose plan matches and a dose plan prediction. The dose prediction module 122 generates the dose plan prediction for the current patient using a dose plan prediction model. The dose plan prediction model will be discussed in further detail with regards to FIGS. 2-3. Additionally, the dose prediction module 122 determines a plurality of previously-administered dose plans using the dose plan prediction of the current patient and the generated features associated with the current patient received from the feature generation module 120. The dose prediction module 122 compares the generated features of the current patient with the generated features of prior patients, as stored in a data store, to identify previously-administered dose plans within a threshold difference of the dose plan prediction. Those within the threshold difference are referred to as the dose plan matches. In the embodiment of FIG. 1, each dose plan match specifies the dose of radiation treatment previously-administered to one or more tumor volumes and surrounding anatomical structures. The plurality of dose plan matches and the dose plan prediction are input as a set of recommended dose plans into the treatment results module 124. In another embodiment, the clinician manually specifies the radiation dose to be delivered to the tumor volume without a prediction by the dose prediction module 122, and this is used as the dose plan prediction and is used to find dose plan matches with prior patients. This dose plan prediction and the dose plan matches are input as a set of recommended dose plans into the treatment results module 124.

The treatment results module 124 predicts the treatment results that may result from the recommended dose plans from the dose prediction module 122. The treatment results module 124 may also identify a plurality of treatment results of previously-administered dose plans of prior patients. As previously described, each dose plan specifies the dose of radiation treatment delivered to one or more tumor volumes and surrounding anatomical structures. As a result of delivering doses of radiation treatment to surrounding anatomical structures, damage may occur to these tissues, which may lead to side effects or symptoms. For example, tissue damage to the lungs may lead to pneumonitis (lung inflammation). The resulting tissue damage is defined as the “toxicity” to the anatomical structure. The level of toxicity is typically graded in severity (i.e., a “toxicity grade”) and is measured by a healthcare provider during follow-up visits, performed in conjunction with the patient through self-assessment, and/or via lab tests.

In the embodiment of FIG. 1, a treatment result represents a set of one or more adverse events and a corresponding outcome for each adverse event that may result from a radiation treatment plan. Examples of an adverse event include myelitis, rib fracture, pericarditis, pneumonitis, or any other type of tissue damage. Each adverse event is associated with one or more of the following outcomes: a probability of level of toxicity to each identified anatomical structure, and a highest probable level of toxicity to each identified anatomical structure. The level of toxicity may be referred to as a “toxicity grade.” As an example, the treatment results module 124 may predict an adverse event of pneumonitis that may have a 30% probability of being grade 3 pneumonitis (on a toxicity grade scale of 1-4, with 4 indicating the highest toxicity grade level). In various embodiments, the scale may vary.

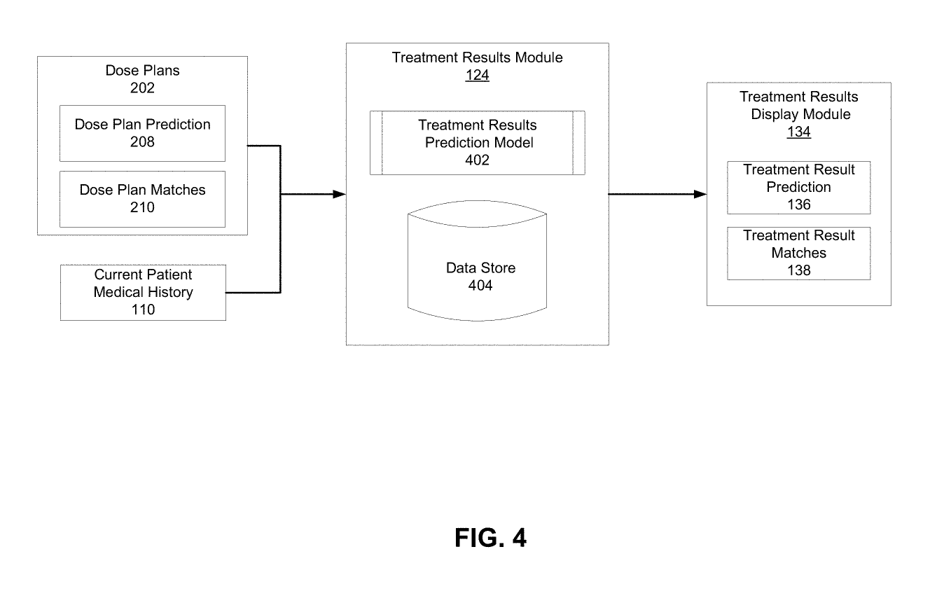

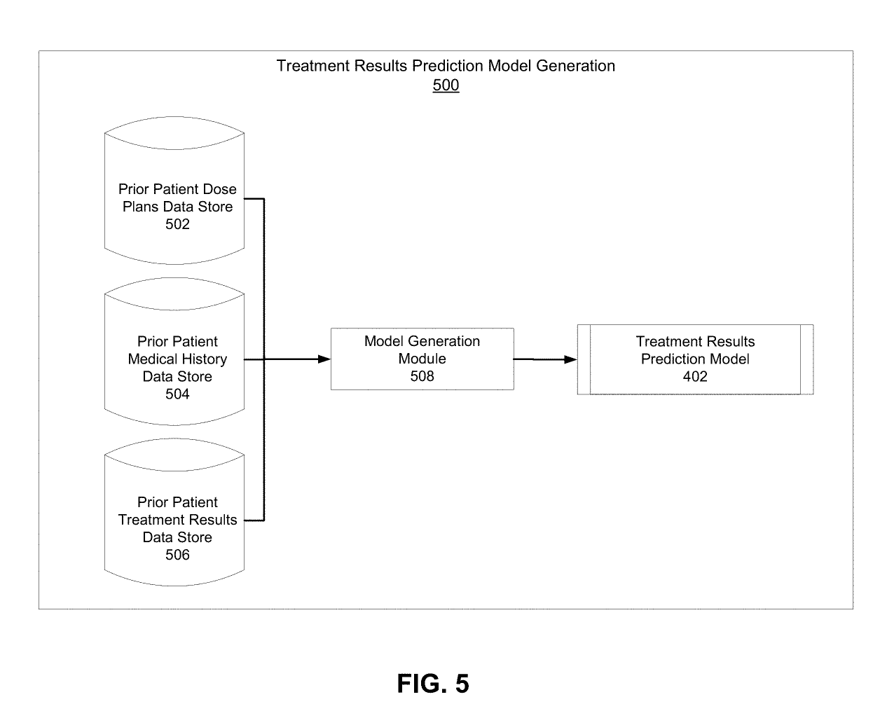

Similar to the dose prediction module 122, the treatment results module 124 outputs a generated set of treatment results. In the embodiment of FIG. 1, the generated set of treatment results may include a plurality of treatment result matches, a treatment result prediction, or some combination thereof. The treatment results module 124 generates the treatment result prediction for the current patient using a treatment results prediction model. The treatment results prediction model will be discussed in further detail with regards to FIGS. 4-5. Additionally, the treatment results module 124 may determine a plurality of treatment results of previously-administered dose plans of prior patients using the treatment result prediction of the current patient and the set of recommended dose plans received from the dose prediction module 122. The treatment results module 124 compares the set of recommended dose plans with previously-administered dose plans of prior patients, as stored in a data store, to identify treatment results of previously administered dose plans of prior patients that are within a threshold difference of the treatment result prediction. Those within the threshold difference are referred to as the treatment result matches. In some embodiments, the treatment results module 124 may determine the plurality of treatment results of previously-administered dose plans of prior patients by using data associated with the current patient (e.g., current patient data 108 and/or current patient medical history 110). The treatment results module 124 compares data associated with the current patient to that of prior patients, as stored in a data store, to identify prior patients having matching or similar data, within a threshold difference, to the current patient. For example, the treatment results module 124 may identify prior patients having a similar medical history to the current patient. Each identified prior patient is associated with a previously-administered dose plan and the associated treatment results, i.e., a treatment result match. The generated set of treatment results, which may include the plurality of treatment result matches, the treatment result prediction, or some combination thereof, are sent to the user interface module 126 for analysis by a clinician.

In some embodiments, the treatment results module 124 may factor in the current patient medical history 110 to improve the precision of the predicted treatment results and the treatment result matches. As previously described, the current patient medical history 110 may include available therapeutic options, especially considering ability to pay for available therapies, genetic results, digital genetics, in vitro lab results, prior treatment history, comorbidities, etc. For example, the treatment results module 124 may predict for a patient who smokes cigarettes an adverse event of pneumonitis with a 70% probability of grade 3 pneumonitis, rather than a 30% probability for a similar patient who doesn't smoke cigarettes. In some embodiments, a treatment result may also include one or more of the following: a probability of tumor recurrence, a type of tumor recurrence such as local recurrence or distant recurrence, and a probable length of time before tumor recurrence.

The user interface module 126 presents the generated set of treatment results for analysis by a clinician. In the embodiment of FIG. 1, the user interface module 126 allows a clinician to visualize, interact with, and modify imaging data of the current patient and the generated set of treatment results to achieve an optimal treatment result for the current patient. As illustrated in the embodiment of FIG. 1, the user interface module 126 includes a visualization module 130 and a treatment results display module 132.

The visualization module 130 displays imaging data of the current patient. In the embodiment of FIG. 1, the visualization module 130 imports the current patient images 112 and the associated current patient contours 114. As previously described, the current patient images 112 may be produced by a medical imaging technology, such as computed tomography (CT), magnetic resonance imaging (MRI), X-ray, fluoroscopy, ultrasound, nuclear medicine including positron emission tomography (PET), or any other suitable medical imaging technology. In the embodiment of FIG. 1, the visualization module 130 includes a contour definition module 134.

The contour definition module 134 is a contouring interface that allows a clinician to view and modify the current patient contours 114 as desired. As previously described, the current patient contours 114 are a set of contours that identify the three-dimensional tumor volumes and the anatomical structures located in the surrounding region of the tumor volumes that are captured in the current patient images 112. The contour definition module 134 overlays the plurality of contours onto the current patient images 112 for concurrent display in the visualization module 130. In the embodiment of FIG. 1, the contour definition module 134 is further configured to allow the clinician to add, delete, and/or modify the current patient contours 114. As a result, the contour definition module 134 creates a new set of current patient contours 114 that includes the added contours, the modified contours, and any remaining un-modified contours that haven't been deleted. The visualization module 130 is synced with the treatment results display module 132 such that modification of the contours in the visualization module 130 may cause the generated set of treatment results to update in the treatment results display module 132.

The treatment results display module 132 displays the generated set of treatment results that are determined by the treatment results module 124. As previously described, the generated set of treatment results may include the plurality of treatment result matches, the treatment result prediction, or some combination thereof. The treatment results display module 132 is populated by the treatment results module 124 and allows a clinician to visualize and compare the generated set of treatment results, as the generated set of treatment results may be effective and/or acceptable treatment results for the patient who is to receive the radiation treatment plan. In some embodiments, the treatment results display module 132 may allow a clinician to input one or more criteria that the treatment results module 124 may use to identify or further narrow treatment result matches of prior patients. For example, the one or more criteria may be identifiers or features from a patient's data or medical history. The input criteria may also allow the clinician to navigate the identified treatment result matches of prior patients by filtering the identified treatment result matches based on the criteria. In the embodiment of FIG. 1, the generated set of treatment results displayed in the treatment results display module 132 are based on the current patient images 112 and the current patient contours 114 that are simultaneously displayed in the visualization module 130. The treatment results display module 132 includes a first portion that is dedicated to displaying the treatment result prediction 136 and a second portion that is dedicated to displaying the treatment result matches 138. The clinician may analyze each of the treatment results to determine if any of the options are optimal treatment results for the current patient. In the embodiment of FIG. 1, the treatment results display module 132 allows the clinician to select one of the displayed treatment results for modification or further optimization.

By simultaneously displaying the visualization module and the treatment results display module 132, a clinician may visualize the relationship between radiation delivery to the current patient contours 114 and the resulting generated set of treatment results. The clinician is able to adjust contours and in real-time evaluate the impact of the tumor treatment and the toxicity risk to the nearby anatomical structures. This configuration allows the clinician to make informed decisions about certain trade-offs during radiation treatment planning for the current patient. In the embodiment of FIG. 1, the clinician may interact with the visualization module 130 to modify the current patient contours 114, or the clinician may interact with the treatment results display module 132 to select a treatment result for optimization, allowing the clinician to create a treatment plan with optimal treatment results.

In the event that the clinician modifies the current patient contours 114 using the contour definition module 134 of the visualization module 130, the contour definition module 134 creates a new set of current patient contours 114 that includes the added contours, the modified contours, and any remaining un-modified contours that haven't been deleted. In the embodiment of FIG. 1, the new set of current patient contours 114 is sent to the feature generation module 120. As a result, the feature generation module 120 generates a new set of features associated with the current patient based on the current patient images 112, the new set of current patient contours 114, the physics parameters 116, and the prescription parameters 118. Subsequently, the dose prediction module 122 generates a new set of recommended dose plans based on the new set of generated features, the treatment results module 124 generates a new set of treatment results based on the new set of recommended dose plans and the current patient medical history 110, and the treatment results display module 132 is updated to display the new set of treatment results. The clinician may analyze the new set of treatment results to determine if any of the options are optimal treatment results for the current patient.

In the event that the clinician selects a treatment result in the treatment results display module 132 for optimization, the selected treatment result and a set of filtering criteria are sent to the treatment result match navigation module 128. In the embodiment of FIG. 1, the set of filtering criteria is defined by the clinician in the treatment results display module 132. The clinician may define the filtering criteria to exclude an adverse event from the treatment result or to place a threshold limit on an outcome of an adverse event. The treatment result match navigation module 128 uses the selected treatment result and the set of filtering criteria to search through a data store of prior patient treatment results to identify a new set of treatment result matches for the current patient. Each of the new treatment result matches is associated with prior patients similar to the current patient. In the embodiment of FIG. 1, the treatment result match navigation module 128 sends the new set of treatment result matches to the treatment results matches 138 for display in the treatment results display module 132. The clinician may analyze the new set of treatment results to determine if any of the options are optimal treatment results for the current patient. In the embodiment of FIG. 1, the clinician may go through a cycle of modifying contours and optimizing a selected treatment result, repeating the steps in any order and as many times as desired, until an optimal treatment result is selected for the current patient.

The treatment delivery engine 106 enables a treatment planner to create a patient-specific treatment delivery plan based on the treatment result selected by the clinician in the treatment planning engine 104. In the embodiment of FIG. 1, the selected treatment result informs the treatment planner of the clinically appropriate profile of radiation to be delivered including but not limited to radiation intensity, angle of delivery, multi-leaf collimator status, temporal fractionation, anatomy, presentation of the tumor, anticipated treatment results, prior clinical staff, and anticipated treatment course.

Based on the treatment delivery plan, the treatment delivery engine 106 generates a patient-specific delivery template that configures a radiation therapy machine for delivering the radiation treatment to the patient. In one embodiment, the treatment delivery engine 106 interacts with a therapy machine control interface that is configured with standard communication protocols. The patient-specific delivery template identifies the tumor volumes as well as the anatomical structures that are to receive radiation treatment. For each volume or structure, the delivery template may also specify the percentage volume that is to receive radiation treatment and the dose of treatment to be delivered. In addition, this template may specify the optimization objects, treatment protocols, beam orientations, collimator/multi-leaf collimator positions, couch positions, and other parameters known in the art.

FIG. 2 illustrates a block diagram of a system for determining the recommended dose plans that are output by the dose prediction module 122, according to an embodiment. As previously described, a dose plan specifies the dose of radiation treatment delivered to one or more tumor volumes and surrounding anatomical structures. As illustrated in FIG. 2, the system includes the current patient data 108, the feature generation module 120, the dose prediction module 122, and dose plans 202.

As described with regards to FIG. 1, the current patient data 108 is data associated with a current patient that is used to generate features associated with the current patient. In the embodiment of FIG. 2, the current patient data 108 includes current patient images 112, current patient contours 114, physics parameters 116, and prescription parameters 118. The current patient data 108 is input into the feature generation module 120 to generate features associated with the current patient.

The dose prediction module 122 predicts the radiation dose delivered to tumor volumes and surrounding anatomical structures. As previously described, the dose prediction module 122 uses the generated features of the current patient received from the feature generation module 120 to determine a set of recommended dose plans. In the embodiment of FIG. 2, the dose prediction module 122 includes a dose plan prediction model 204 and a data store 206. The dose prediction module 122 outputs dose plans 202, which is a set of recommended dose plans including a dose plan prediction 208 and a plurality of dose plan matches 210.

The dose plan prediction model 204 is a prediction model used to generate the dose plan prediction 208. In the embodiment of FIG. 2, the dose plan prediction model 204 is created using historical data of prior patients that have received radiation treatment. FIG. 3 illustrates a system environment 300 for generating a radiation dose plan prediction model, according to an embodiment. As illustrated, the dose plan prediction model generation system 300 includes prior patient data 302, a feature generation module 304, a model generation module 306, and a prior patient dose plans data store 308 to generate the dose plan prediction model 204.

The prior patient data 302 is data associated with a prior patient that is used to generate features associated with the prior patient. Similar to the current patient data 108, in the embodiment of FIG. 3, the prior patient data 302 includes prior patient images 310, prior patient contours 312, physics parameters 314, and prescription parameters 316. Similarly, the characteristics of the current patient images 112, the current patient contours 114, the physics parameters 116, and the prescription parameters 118 may be incorporated herein for the prior patient images 310, the prior patient contours 312, the physics parameters 314, and the prescription parameters 316, respectively. The prior patient data 302 may be historical data gathered from databases from a plurality of hospitals, clinics, cancer treatment centers, or any other center for radiation therapy. In addition to gathering the prior patient data 302, historical data regarding dose plans for each prior patient is gathered as well. In the embodiment of FIG. 3, the prior patient data 302, once gathered from other databases, may be stored in the prior patient data store 307. The prior patient dose plans may be stored the prior patient dose plans data store 308 in three-dimensional point sets or in dose volume histograms (DVHs).

The feature generation module 304 generates features associated with a prior patient. In the embodiment of FIG. 3, the feature generation module 304 imports the prior patient data 302, which includes prior patient images 310, prior patient contours 312, physics parameters 314, and prescription parameters 316. For a plurality of the prior patients stored in the prior patient data store 307, the feature generation module 304 generates a set of features associated with a specific prior patient using the prior patient's respective prior patients feature data 302. Similar to the generated features of current patient as described with regards to FIG. 1, these generated features embody the relationship between radiation delivery and dose. For each prior patient, the set of generated features may be stored in the prior patient data store 307. In some embodiments, the feature generation module 304 is functionally the same as the feature generation module 120.

The model generation module 306 generates the dose plan prediction model 204 by using the stored data of each prior patient in the prior patient data store 307 and the prior patient dose plans data store 308. In the embodiment of FIG. 3, the model generation module 306 accesses the set of generated features and prior patient dose plans for each prior patient and performs a regression analysis to create the dose plan prediction model 204 that relates the generated features to the dose plans. The dose plan prediction model 204 may be saved within the dose prediction module 122 such that the dose plan prediction model 204 may be used innumerous times to generate dose plan predictions for a plurality of patients to receive radiation treatment. The dose plan prediction model 204 may also be updated through machine-learning techniques and as additional prior patient data 302 and associated dose plans are gathered from other databases.

Returning to FIG. 2, the dose plan prediction model 204 may be used to generate the dose plan prediction 208 for a current patient. The generated features of the current patient from the feature generation module 120 are used as inputs into the dose plan prediction model 204. Since the dose plan prediction 208 is generated using a prediction model, the dose plan prediction 208 may not be a previously-administered dose plan of a prior patient. In some embodiments, the dose plan prediction 208 generated by the dose plan prediction model 204 is stored as a historical data point within the data store 206 and may be used as an additional data point to update the dose plan prediction model 204.

The data store 206 is a data store of previously-administered dose plans of prior patients and generated features associated with prior patients. In some embodiments, the data store 206 may be the same data store as the prior patient data store 307, the prior patient dose plans data store 308, or the data store 206 may combine the data stored within the data stores 307 and 308. In other embodiments, the data store 206 may be created once the generated features of the current patient are input into the dose plan prediction model 204 and a set of dose plans of prior patients from the prior patient dose plans data store 308 may be identified that are similar to the resulting dose plan prediction 208.

In the embodiment of FIG. 2, the dose prediction module 122 accesses the data store 206 to retrieve dose plans and generated features associated with prior patients. The dose prediction module 122 performs a multi-feature comparative analysis between a) the generated features associated with the current patient and the dose plan prediction 208, and b) the generated features associated with prior patients to identify one or more previously-administered dose plans of the prior patients that are within a threshold difference of the dose plan prediction 208. The identified previously-administered dose plans may be the most suitable for the current patient, referred to as the dose plan matches 210. Alternatively, the dose prediction module 122 may identify a subset of similar prior patients based on the generated features, herein referred to as a “dose plan neighborhood.” The dose plan neighborhood may beneficially inform the clinician of a variety of dose plans based on prior patient contours, radiation delivery technology, and/or different treatment therapies. In the embodiment of FIG. 2, once the dose plan prediction model 204 generates the dose plan prediction 208 and the dose prediction module 122 identifies the dose plan matches 210, the dose prediction module 122 outputs the dose plans 202 to the treatment results module 124.

FIG. 4 illustrates a block diagram of a system for generating a set of treatment results that are output by the treatment results module 124, according to an embodiment. As previously described, a treatment result of a treatment plan represents a set of one or more adverse events and a corresponding outcome for each adverse event that may result from radiation delivered to an anatomical structure. In the embodiment of FIG. 4, the system includes the dose plans 202, the current patient medical history 110, the treatment results module 124, and the treatment results display module 134.

As described with regards to FIG. 2, the dose plans 202 are a set of recommended dose plans including the dose plan prediction 208 and the plurality of dose plan matches 210. The dose plans 202 and the current patient medical history 110 are input into the treatment results module 124 to generate a set of treatment results.

The treatment results module 124 predicts the treatment results that may result from the recommended dose plans that are determined by the dose prediction module 122. The treatment results module 124 may also identify a plurality of treatment results of previously-administered dose plans of prior patients. For example, the treatment results module may provide the treatment results associated with the dose plan matches 210 identified by the dose prediction module 122. In some embodiments, the treatment results module 124 may be configured to perform one or both of these functionalities. As previously described, the treatment results module 124 uses the dose plans 202 and the current patient medical history 110 to generate a set of treatment results, which may include a plurality of treatment result matches, a treatment result prediction, or some combination thereof. In the embodiment of FIG. 4, the treatment results module 124 includes a treatment results prediction model 402 and a treatment results data store 206, but the modules may vary in other embodiments. The treatment results module 124 outputs the treatment result prediction 136, the plurality of treatment result matches 138, or some combination thereof for display in the treatment results display module 134.

The treatment results prediction model 402 is a prediction model used to generate the treatment result prediction 136. In the embodiment of FIG. 4, the treatment results prediction model is created using historical data of prior patients that have received radiation treatment. FIG. 5 illustrates a system environment 500 for generating a treatment results prediction model 402, according to an embodiment. As illustrated, the treatment results prediction model generation system 500 includes a prior patient dose plans data store 502, a prior patient medical history data store 504, a prior patient treatment results data store 506, and a model generation module 508 to generate the treatment results prediction model 402.

The prior patient dose plans data store 502 is a data store of previously-administered dose plans of prior patients. In some embodiments, the prior patient dose plans data store 502 may be the same data store as the prior patient dose plans data store 308. The prior patient medical history data store 504 is a data store of medical history of prior patients. As previously described, medical history may include available therapeutic options, especially considering ability to pay for available therapies, genetic results, digital genetics, in vitro lab results, prior treatment history, comorbidities, etc. The prior patient treatment results data store 506 is a data store of treatment results of previously-administered dose plans of prior patients. The prior patient data stored in data stores 502, 504, and 506 may be historical data gathered from databases from a plurality of hospitals, clinics, cancer treatment centers, or any other center for radiation therapy. The prior patient dose plans may be stored in the prior patient dose plans data store 502 in three-dimensional point sets or in dose volume histograms (DVHs).

The model generation module 508 generates the treatment results prediction model 402 by using the stored data of each prior patient in the prior patient dose plans data store 502, the prior patient medical history data store 504, and the prior patient treatment results data store 506. In the embodiment of FIG. 5, the model generation module 508 accesses the dose plans, medical history, and treatment results for each prior patient and performs a regression analysis to create the treatment results prediction model 402 that relates the dose plans and medical history to the treatment results. The treatment results prediction model 402 may be saved within the treatment results prediction module 124 such that the treatment results prediction model 402 may be used innumerous times to generate treatment result predictions for a plurality of patients to receive radiation treatment. The treatment results prediction model 402 may also be updated through machine-learning techniques and as additional prior patient data is gathered from other databases.

Returning to FIG. 4, the treatment results prediction model 402 may be used to predict the treatment result prediction 136 for a current patient. The treatment results prediction model 402 receives the dose plans 202 and the current patient medical history 110 as inputs into the treatment results prediction model 402. Since the treatment result prediction 136 is generated using a prediction model, the treatment result prediction 136 may not be a treatment result of a previously-administered dose plan of a prior patient. In some embodiments, the treatment result prediction 136 generated by the treatment results prediction model 402 is stored as a historical data point within the data store 404 and may be used as an additional data point to update the treatment results prediction model 402.

The data store 404 is a data store of treatment results of previously-administered dose plans of prior patients, previously-administered dose plans of prior patients, and prior patient medical history. In some embodiments, the data store 404 may be the same data store as the prior patient dose plans data store 502, the prior patient medical history data store 504, or the prior patient treatment results data store 506, or the data store 404 may combine the data stored in the three data stores 502, 504, and 506. In the embodiment of FIG. 4, the treatment results module 124 accesses the data store 404 to retrieve treatment results, dose plans, and medical history associated with prior patients. Similar to the dose prediction module 122, the treatment results module 124 performs a multi-feature comparative analysis between a) the dose plans 202, the current patient medical history 110, and the treatment result prediction 136, and b) the previously-administered dose plans of prior patients and prior patient medical history to identify one or more treatment results of previously-administered dose plans of prior patients that are within a threshold difference of the treatment result prediction 136. Alternatively, the treatment results module 124 may identify a subset of similar prior patients based on the dose plans, the medical history, or some combination thereof, herein referred to as a “treatment result neighborhood.” In some embodiments, a clinician may specify one or more criteria that the treatment results module 124 uses to identify the subset of similar prior patients. The specified filter criteria may comprise characteristics from the dose plans, the medical history, or some combination thereof. The treatment result neighborhood may beneficially inform the clinician of a variety of treatment results based on prior patient contours, prior patient dose plans, radiation delivery technology, different treatment therapies, or some combination thereof. The identified treatment results may be the most suitable for the current patient, referred to as the treatment result matches 138. In the embodiment of FIG. 4, once the treatment results prediction model 402 generates the treatment result prediction 136 and the treatment results module 124 identifies the treatment result matches 138, the treatment results module 124 outputs the set of treatment results to the treatment results display module 134 for analysis by a clinician. In alternate embodiments, the treatment results module 124 identifies the treatment result matches 138 and outputs those to the treatment results display module 134 as the generated set of treatment results for display.

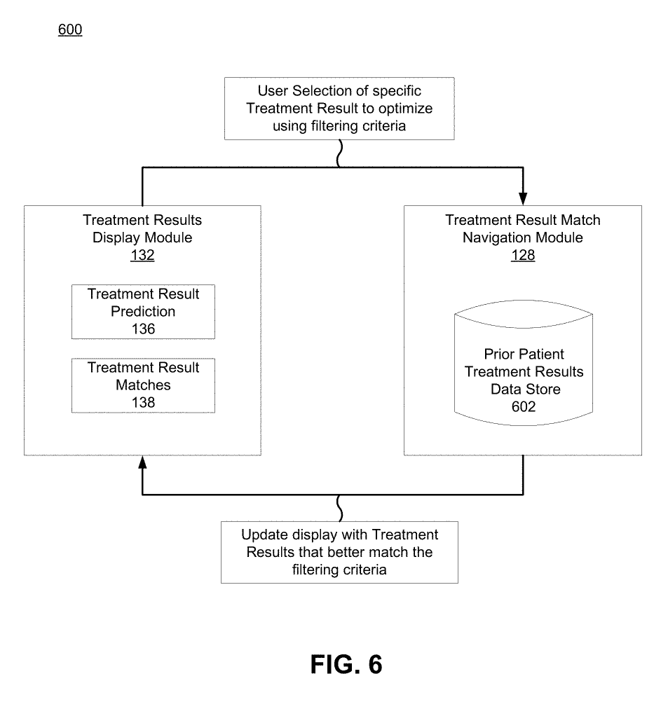

FIG. 6 illustrates a system 600 for optimizing a selected treatment result, according to an embodiment. As illustrated in FIG. 6, the system 600 includes the treatment results display module 132 which includes the treatment result prediction 136 and the treatment result matches 138. In alternate embodiments, the treatment results display module 132 may include one or both. The system 600 further includes the treatment result match navigation module 128 which includes a prior patient treatment results data store 602.

The treatment results display module 132 displays the generated set of treatment results, comprised of the treatment result prediction 136, the plurality of treatment result matches 138, or some combination thereof, that are determined by the treatment results module 124. The treatment results display module 132 allows a clinician to analyze each of the treatment results to determine if any of the options are optimal treatment results for the current patient. In some embodiments, the treatment results display module 132 may allow a clinician to input one or more criteria that the treatment results module 124 may use to identify or further narrow treatment result matches of prior patients. For example, the one or more criteria may be identifiers or features from a patient's data or medical history. The input criteria may also allow the clinician to navigate the identified treatment result matches of prior patients by filtering the identified treatment result matches based on the criteria. In the embodiment of FIG. 6 the treatment results display module 132 allows the clinician to select one of the displayed treatment results for modification or further optimization.

Upon selecting one of the displayed treatment results, the clinician may define, within the treatment results display module 132, one or more filtering criteria with which to optimize the selected treatment result. The clinician may define the filtering criteria to exclude an adverse event from the treatment result or to place a threshold limit on an outcome of an adverse event. For example, the clinician may specify a first filtering criteria to exclude bone fracture as an adverse event and a second filtering criteria to limit pneumonitis to a toxicity grade of level 1 (on a scale of 1-4, with 4 as the most severe). The filtering criteria are input into the treatment result match navigation module 128.

The treatment result match navigation module 128 uses the selected treatment result and the set of filtering criteria to search through the prior patient treatment results data store 602 to identify a new set of treatment result matches 138 for the current patient. Each of the new treatment result matches is associated with prior patients similar to the current patient. The new set of treatment result matches 138 may provide treatment results that are optimized based on the filtering criteria relative to the selected treatment result. In the embodiment of FIG. 6, the treatment result match navigation module 128 sends the new set of treatment result matches to the treatment results matches 138 for display in the treatment results display module 132. The clinician may analyze the new set of treatment results to determine if any of the options are optimal treatment results for the current patient. In the embodiment of FIG. 6, the clinician may go through a cycle of modifying contours and optimizing a selected treatment result, repeating the steps in any order and as many times as desired, until an optimal treatment result is selected for the current patient.

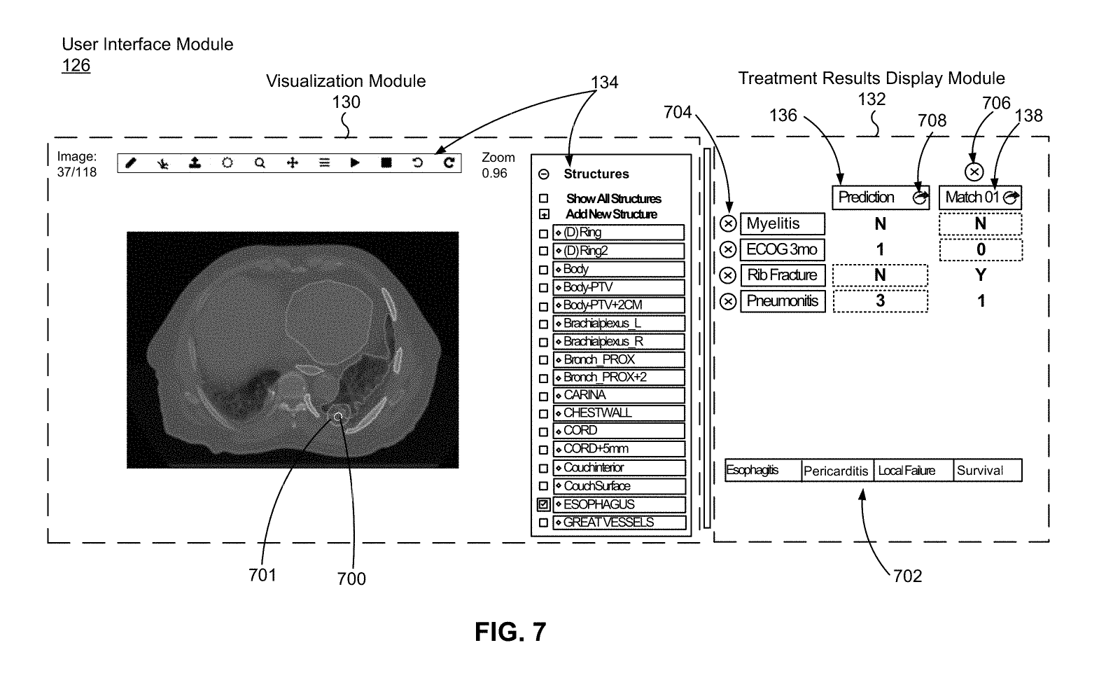

FIG. 7 illustrates an exemplary user interface for visualizing treatment results, according to an embodiment. In the embodiment of FIG. 7, the user interface module 126 simultaneously displays the visualization module 130 with the treatment results display module 132. In the embodiment of FIG. 7, the user interface module 126 allows a clinician to visualize, interact with, and modify imaging data of the current patient and the set of treatment results to achieve an optimal treatment result for the current patient.

The visualization module 130 displays imaging data of the current patient. In the embodiment of FIG. 7, the visualization module 130 imports the current patient images 112 and the corresponding current patient contours 114. As previously described, the current patient images 112 may be produced by a medical imaging technology, such as computed tomography (CT), magnetic resonance imaging (MM), X-ray, fluoroscopy, ultrasound, nuclear medicine including positron emission tomography (PET), or any other suitable medical imaging technology. In the embodiment of FIG. 7, the current patient images 112 may be a plurality of images, wherein each image illustrates the same perpendicular cross-section of the interior of the patient's body but at various depths of the patient's body. The user interface module 126 may be configured to allow a clinician to scroll through the current patient images 112 such that cross-sections at various depths of the patient's body can be displayed in the visualization module 130 with the corresponding contours for each image. In the embodiment of FIG. 1, the visualization module 130 includes a contour definition module 134.

The contour definition module 134 is a contouring interface that allows a clinician to view and modify the current patient contours 114 as desired. As previously described, the current patient contours 114 are a set of contours that identify the three-dimensional tumor volumes and the anatomical structures located in the surrounding region of the tumor volumes that are captured in the current patient images 112. The contours surrounding the one or more tumor volumes indicate the areas towards which a radiation dose is targeted. The contours surrounding the one or more tumor volumes may be defined in view of standard practices relating to gross tumor volume (GTV), clinical tumor volume (CTV), and planning tumor volume (PTV), such that these contours may include portions of surrounding anatomical structures. The contours for the surrounding anatomical structures may highlight either a portion of or all of the anatomical structure. The contour definition module 134 overlays the plurality of contours onto the current patient images 112 for concurrent display in the visualization module 130. As illustrated in FIG. 7, imaging data of the current patient is shown with a plurality of current patient contours 114 in the visualization module 130. Each outline in the image is a contour line, and each contour line can be a different color to represent a different structure. The different colors can be indicated in a key to the right that lists the name of each structure in the current image in the visualization module 130. Using the key, the clinician can quickly identify which contour corresponds to which structure in the list. In the embodiment of FIG. 7, a contour 700 surrounds the gross tumor volume (GTV), a contour 701 surrounds the planning tumor volume (PTV), and the remaining contours highlight neighboring anatomical structures. In some images, various contours may overlap with each other, as shown in FIG. 7. In the embodiment of FIG. 7, the current patient contours 114 are imported with the current patient images 112 such that the contours readily appear with each image displayed in the visualization module 130. In some embodiments, the contour definition module 134 may be configured to automatically generate contours or may provide suggested contours for the current patient images 112.

In the embodiment of FIG. 7, the contour definition module 134 is configured to allow a clinician to add, delete, and/or modify the current patient contours 114. The contour definition module 134 includes a toolbar that allows the clinician to modify existing contours displayed in the visualization module 130. Using the toolbar, the clinician may change the shape of a selected contour. For example, a clinician may wish to modify an existing contour if a contour was initially defined incorrectly or to increase the margin of a contour surrounding a tumor volume. The contour definition module 134 also includes a structures list, which represents the various anatomical structures shown in the displayed current patient image 112. By selecting or de-selecting anatomical structures in the structures list, the contour definition module 134 causes contours for the anatomical structure to appear or disappear from the visualization module 130. The clinician may also add a new contour for an anatomical structure that may not have been initially defined. Adding and creating contours for an anatomical structure in the contour definition module 134 results in the treatment results display module 132 updating to include adverse events associated with the anatomical structure and the corresponding outcomes of the adverse events.

In the embodiment of FIG. 7, the contour definition module 134 is configured to detect if a contour has been added, deleted, and/or modified by a clinician. As a result, the contour definition module 134 captures the new set of current patient contours 114 that includes the added contours, the modified contours, and any remaining un-modified contours that haven't been deleted and sends the new set of current patient contours 114 to the feature generation module 120 so that a new set of treatment results is subsequently generated. In the embodiment of FIG. 7, the visualization module 130 is synced with the treatment results display module 132 such that modification of the contours in the contour definition module 134 cause the generated set of treatment results to update in the treatment results display module 132 in real-time. In some embodiments, the user interface module 126 may require the clinician to indicate when a new set of current patient contours 114 is ready to be captured, such that the treatment results display module 132 can be updated accordingly.

The treatment results display module 132 displays the set of treatment results that are generated by the treatment results module 124. As previously described, the generated set of treatment results may include the plurality of treatment result matches, the treatment result prediction, or some combination thereof. The treatment results display module 132 is populated by the treatment results module 124 and allows a clinician to visualize and compare the generated set of treatment results. In some embodiments, the treatment results display module 132 may allow a clinician to input one or more criteria that the treatment results module 124 may use to identify or further narrow treatment result matches of prior patients. In the embodiment of FIG. 1, the generated set of treatment results displayed in the treatment results display module 132 are based on the current patient images 112 and the current patient contours 114 that are simultaneously displayed in the visualization module 130. The clinician may analyze each of the treatment results to determine if any of the options are optimal treatment results for the current patient.

In the embodiment of FIG. 7, the treatment results display module 132 presents the generated set of treatment results in a table format. As previously described, the generated set of treatment results may include the plurality of treatment result matches, the treatment result prediction, or some combination thereof. Each treatment result represents a set of one or more adverse events and a corresponding outcome for each adverse event that may result from a radiation treatment plan. As illustrated in FIG. 7, each column heading labels a treatment result, while each row labels an adverse event associated with the current patient contours 114 in the visualization module 130. Examples of adverse events, as shown in FIG. 7, may include “Myelitis,” “ECOG 3mo” (a physician's assessment of a patient's performance status), “Rib Fracture,” and “Pneumonitis.” In some embodiments, a column labeled “Prediction” displays the treatment result prediction 136, listing the predicted outcome for each adverse event. The neighboring column labeled “Match 01” displays a first treatment result match 138, listing an outcome for each adverse event that resulted from a previously-administered treatment plan of a prior patient. The treatment results display module 132 may display multiple neighboring columns if there are multiple treatment result matches 138, e.g. “Match 02,” “Match 03,” etc. In some embodiments, the treatment results display module 132 may limit the number of treatment result matches 138 to avoid displaying less similar treatment result matches. The treatment results display module 132 may additionally include a text field, drop-down menu, or a similar variation that allows a clinician to input or select one or more criteria for identifying or further narrowing treatment result matches of prior patients. Displaying the treatment result prediction 136 with the treatment result matches 138 may allow a clinician to compare the treatment result prediction 136 to the treatment result matches 138 to determine that the treatment result prediction 136 is a reasonable treatment result and that the clinician may accurately expect the treatment result as predicted, serving as a confidence check for the clinician.

In the embodiment of FIG. 7, each element of the table describes an outcome of an adverse event of a corresponding treatment result. Each adverse event is associated with one or more of the following outcomes: a probability of level of toxicity to each identified anatomical structure, and a highest probable level of toxicity to each identified anatomical structure. The level of toxicity may be referred to as a “toxicity grade.” In the embodiment of FIG. 7, for ease of display, only the highest probable toxicity grade may be displayed for each adverse event. For example, for the treatment result prediction 136, the adverse event “Pneumonitis” has an outcome of “3,” indicating that toxicity grade 3 pneumonitis is more likely to occur than any other toxicity grade of pneumonitis. For some adverse events, the adverse event may have a binary outcome, such as “Yes, adverse event will occur,” or “No, adverse event will not occur.” For example, for the treatment result match 138, the adverse event “Rib Fracture” has an outcome of “Y,” indicating that a rib fracture occurred as a result of the treatment plan for this prior patient. Other adverse events may be characterized by a level of function rather than a level of damage. For example, the adverse event “ECOG 3mo” is a physician's assessment of a patient's overall performance status at three months post-therapy. As shown in FIG. 7, for the treatment result prediction 136, “ECOG 3mo” has an outcome of “1,” indicating functional level 1 (on a scale of 0-5, with 0 as the highest functional level). In various embodiments, the scale may vary. In some embodiments, the treatment results display module 132 may also display treatment efficacy for each treatment result, such as a probability of tumor recurrence, a type of tumor recurrence such as local recurrence or distant recurrence, and a probable length of time before tumor recurrence. In the embodiment of FIG. 7, the outcomes may be emphasized in various colors or with various markings to indicate preferred outcomes when comparing outcomes of adverse events between treatment results. As illustrated in FIG. 7, an outcome in a dashed box may be preferred over an outcome without a dashed box (e.g., “No rib fracture” is a preferable outcome over “Yes, rib fracture”). Various outcomes may also be emphasized with a specific color or maring to indicate an unacceptable outcome, which may be clinician-specific.

The treatment results display module 132 may include a variety of buttons that enable a clinician to modify the information shown in the treatment results display module 132. These modifications may allow the clinician to display only information that is deemed relevant or important to the current patient, facilitating an efficient, streamlined treatment planning process for the clinician. In the embodiment of FIG. 7, the buttons include a plurality of adverse event buttons 702, a plurality of adverse event removal buttons 704, a plurality of treatment result removal buttons 706, and a plurality of treatment result optimization buttons 708.

The plurality of adverse event buttons 702 represent additional adverse events that can be considered as a part of the treatment results. In the embodiment of FIG. 7, the plurality of adverse event buttons 702 may be located below the table of treatment results. A clinician may select one or more of the adverse event buttons if the clinician would like to determine the corresponding outcome of the adverse event for each treatment result. When an adverse event button is selected, that adverse event is added as an additional row in the table of treatment results, and the corresponding outcome of the adverse event for each treatment result will populate the elements of the row. In some embodiments, the plurality of adverse event buttons 702 may auto-populate with adverse events that may be relevant to the current patient images 112 and the current patient contours 114 as a clinician scrolls through each patent image. A clinician may also manually add an adverse event to the table of treatment results if the adverse event doesn't appear on an adverse event button 702. In some embodiments, an adverse event button may be emphasized (e.g., highlighted, flashing, or the like) in the treatment results display module 132 if the system 100 detects that a corresponding outcome of the adverse event is above a threshold limit, indicating that the adverse event may be significant and should be considered as a part of the treatment results.

The plurality of adverse event removal buttons 704 allow a clinician to remove adverse events from the table of treatment results. In the embodiment of FIG. 7, an adverse event removal button 704 is located next to the row of each adverse event. When selected, the entire row associated with the adverse event is removed from the table. A clinician may select to remove an adverse event if the adverse event is deemed irrelevant or is not a concern for the current patient. By removing unnecessary adverse events from the treatment results display module 132, the clinician is able to focus on the important information to determine an optimal treatment result for the current patient.

The plurality of treatment result removal buttons 706 allow a clinician to remove treatment results from the table of treatment results. In the embodiment of FIG. 7, a treatment result removal button 706 is located above the column of each treatment result match 138. When selected, the entire column associated with the treatment result is removed from the table. A clinician may select to remove a treatment result if the clinician has determined that alternate treatment results are more suitable for the current patient. By removing unnecessary treatment results from the treatment results display module 132, the clinician is able to more efficiently narrow down the treatment results to determine an optimal treatment result for the current patient. In some embodiments, the treatment result prediction 136 may or may not be removed by a treatment result removal button 706. In some embodiments, the treatment results display module 132 is configured to only display a plurality of the treatment result matches 138.

The plurality of treatment result optimization buttons 708 allows a clinician to select a treatment result for optimization. In the embodiment of FIG. 7, a treatment result optimization button 708 is located next to the column heading of each treatment result. A clinician may select a treatment result optimization button 708 for a treatment result if the clinician believes the selected treatment result can be modified to achieve an optimal treatment result for the current patient. When a treatment result is selected for optimization, the treatment results display module allows the clinician to define a plurality of filtering criteria, such as excluding an adverse event from the treatment result or to place a threshold limit on an outcome of an adverse event. For example, the clinician may specify a first filtering criteria to exclude “Rib Fracture” as an adverse event and a second filtering criteria to limit “Pneumonitis” to a toxicity grade of level 1 (for example, on a scale of 1-4, with 4 as the most severe). The selected treatment result and the filtering criteria are input into the treatment result match navigation module 128 to identify a new set of treatment result matches 138 for the current patient. Subsequently, the treatment result match navigation module 128 sends the new set of treatment result matches to the treatment results matches 138 for display in the treatment results display module 132. The clinician may analyze the new set of treatment results to determine if any of the options are optimal treatment results for the current patient. In some embodiments, if the clinician determines that one of the treatment results is optimal for the current patient, the clinician may select the treatment result optimization button 708 of the treatment result to export the selected treatment result to the treatment delivery engine 106 to generate a patient-specific treatment delivery plan based on the selected treatment result.

By simultaneously displaying the visualization module and the treatment results display module 132, a clinician may visualize the relationship between radiation delivery to the current patient contours 114 and the resulting treatment results. Using the user interface module 126, the clinician is able to adjust contours and in real-time evaluate the impact on the tumor volume and the toxicity risk to the nearby anatomical structures. The clinician may go through a cycle of scrolling through each of the plurality of current patient images 112 to modify contours, add or remove adverse events from consideration, add or remove treatment results from consideration, and optimize a selected treatment result until the clinician determines an optimal treatment result for the current patient. This configuration enables the clinician to make informed decisions about certain trade-offs during radiation treatment planning.

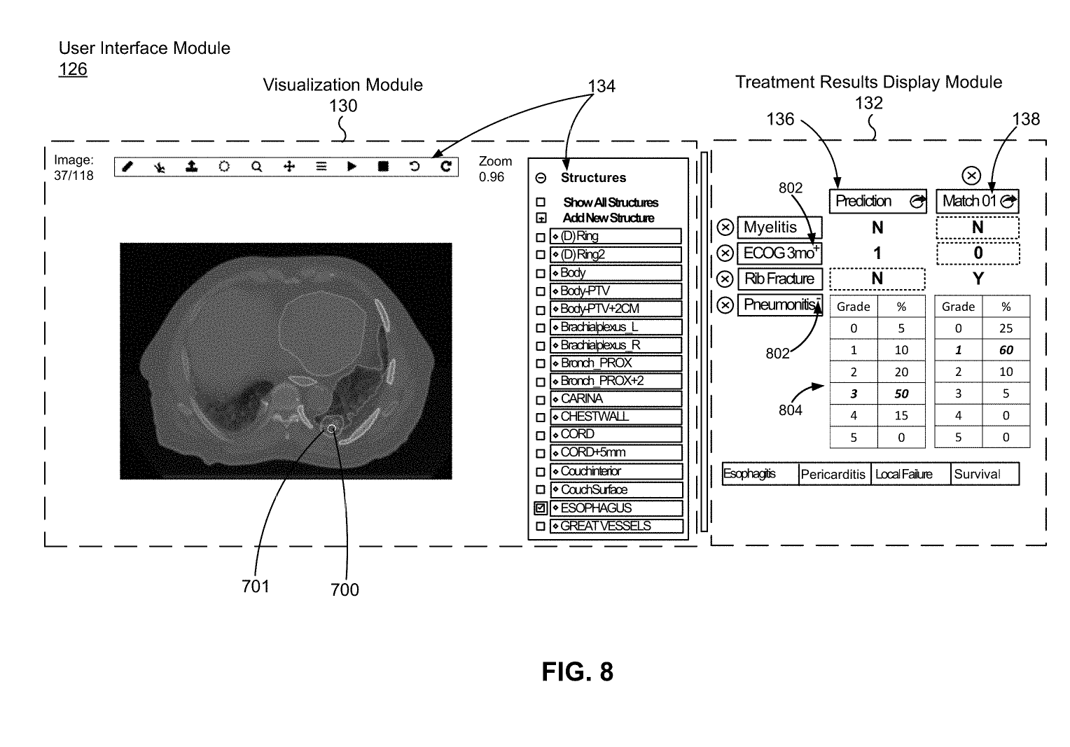

FIG. 8 illustrates an exemplary user interface for visualizing treatment results, according to an additional embodiment. As previously described, the generated set of treatment results may include the plurality of treatment result matches, the treatment result prediction, or some combination thereof. As described with regards to FIG. 7, each element of the table describes an outcome of an adverse event of a corresponding treatment result. Each adverse event is associated with one or more of the following outcomes: a probability of level of toxicity to each identified anatomical structure, and a highest probable level of toxicity to each identified anatomical structure. In the embodiment of FIG. 7, for ease of display, only the highest probable toxicity grade may be displayed for each adverse event. In the embodiment of FIG. 8, an adverse event may include a toggle button 802 next to the row heading. Selecting the toggle button 802 causes an outcome table, such as outcome table 804, to expand for the adverse event for each treatment result. As illustrated in FIG. 8, the outcome table 804 for “Pneumonitis” displays the toxicity grades and the corresponding probability for each toxicity grade. The toxicity grade with the highest probable level of toxicity may be emphasized in the outcome table 804. For example, for the treatment result prediction 136, toxicity grade 3 pneumonitis has a 50% probability of occurring as a treatment result, which is higher than the probabilities of the remaining toxicity grades. Thus, as described with regards to FIG. 7, for ease of display, only toxicity grade 3 is displayed in the treatment results display module 132 when the toggle button 802 is selected to minimize the outcome table 804. In the embodiment of FIG. 8, only adverse events that have corresponding outcomes related to toxicity grades may have a toggle button 802.

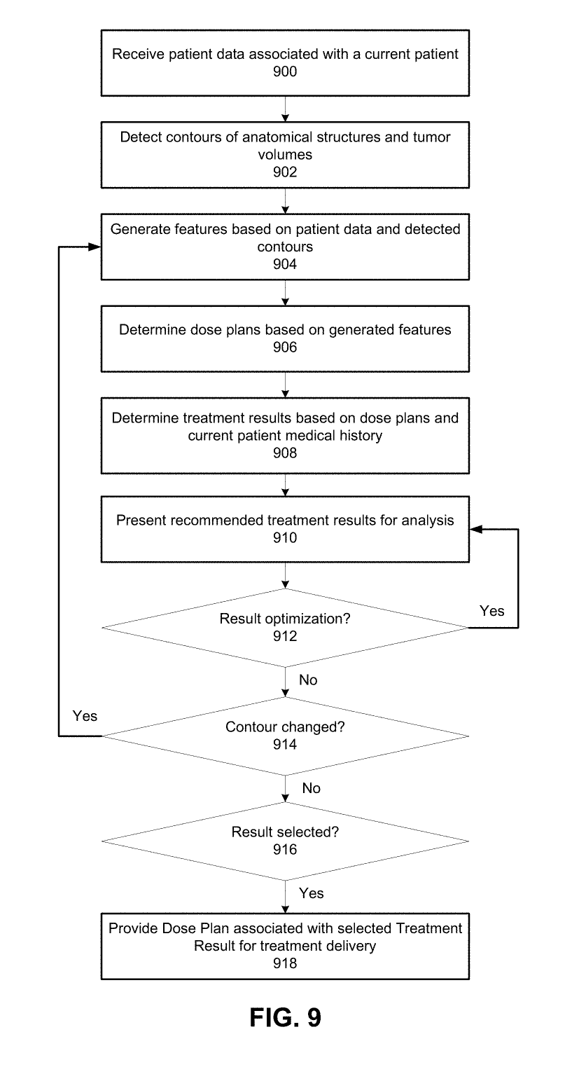

FIG. 9 illustrates a flow diagram illustrating the steps for clinician-directed radiation treatment planning, according to an embodiment. The flow diagram represents a workflow for creating a radiation treatment plan using system 100, as described with regards to FIG. 1.

As described in 900, a patient data engine receives patient data associated with a current patient. The current patient data may include patient images, patient contours, physics parameters, and prescription parameters. The current patient data is input into a treatment planning engine.

As described in 902, the treatment planning engine detects contours of anatomical structures and tumor volumes in the current patient data. These contours may be defined in each patient image by a clinician before the current patient data is imported into the system.

As described in 904, the treatment planning engine generates features associated with the current patient based on the current patient data and the detected contours. The generated features can be compared to similar generated features of prior patients to identify prior patients that are similar to the current patient.

As described in 906, the treatment planning engine determines a plurality of dose plans. The dose plans include a dose plan prediction and a plurality of dose plan matches. To determine the dose plan prediction, the treatment planning engine inputs the generated features of the current patient into a dose plan prediction model, which generates the dose plan prediction. To determine the plurality of dose plan matches, the treatment planning engine uses the dose plan prediction and the generated features of the current patient. The treatment planning engine compares the generated features of the current patient with generated features of prior patients to identify previously-administered dose plans of prior patients that are similar to the current patient. The treatment planning engine then determines which of the previously-administered dose plans are within a threshold difference of the dose plan prediction. Those within the threshold difference are the dose plan matches. The plurality of dose plan matches and the dose plan prediction comprise a set of dose plans.

As described in 908, the treatment planning engine determines a plurality of treatment results based on dose plans and current patient medical history. The treatment results include a treatment result prediction and a plurality of treatment result matches. To determine the treatment result prediction, the treatment planning engine inputs the dose plans and the current patient medical history into a treatment results prediction model, which generates the treatment result prediction. To determine the plurality of treatment result matches, the treatment planning engine uses the treatment result prediction, the dose plans, and the current patient medical history. The treatment planning engine compares the dose plans with previously-administered dose plans of prior patients to identify treatment results of previously-administered dose plans of prior patients. The treatment planning engine then determines which of the identified treatment results are within a threshold difference of the treatment result prediction. Those within the threshold difference are the treatment result matches. The plurality of treatment result matches and the treatment result prediction comprise a set of treatment results.

As described in 910, the treatment planning engine presents the treatment results for analysis by a clinician. The generated set of treatment results and the current patient images and the current patient contours on which the treatment results are based are simultaneously displayed. By simultaneously displaying them, the clinician may visualize the relationship between radiation delivery to the current patient contours and the resulting treatment results. The clinician is able to adjust contours and in real-time evaluate the impact on the tumor volume and the toxicity risk to the nearby anatomical structures. This allows the clinician to make informed decisions about certain trade-offs during radiation treatment planning for the current patient. The clinician may modify the current patient contours to generate a new set of treatment results, or the clinician may select a treatment result for optimization, allowing the clinician to create a treatment plan with optimal treatment results.

As described in 912, a clinician may select a treatment result for optimization. If a treatment result is selected, the clinician may define a plurality of filtering criteria. The treatment planning engine accesses a data store to identify a new set of treatment results based on the selected treatment result and the filtering criteria. The new set of treatment results is presented for analysis by the clinician, as discussed in 910.

As described in 914, a clinician is able to modify the current patient contours. If the treatment planning engine detects that a contour is modified, a new set of current patient contours is captured. As a result, 904-914 may be repeated.

As described in 916, a clinician may select a treatment result as an optimal treatment result for the current patient. The selected treatment result is sent to a treatment delivery engine to create a radiation dose treatment plan that will produce the selected treatment result.

As described in 918, the treatment delivery engine provides a radiation dose treatment plan associated with the selected treatment result to a clinician for treatment delivery to the current patient.

### Concluding Comments

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for creating a radiation treatment plan for a given patient, the method comprising:
receiving data associated with a patient who is to receive radiation treatment, wherein the data comprises a medical history of the patient;
generating a plurality of features associated with the patient based on the received data;
providing the plurality of generated features to a prediction model to predict a treatment plan for the patient, wherein the treatment plan comprises a radiation treatment dose to be administered to the patient;
searching a database of previously-administered radiation treatment plans of other patients for treatment plans that are within a threshold difference from the predicted treatment plan, each previously-administered radiation treatment plan including a treatment result;
identifying a set of previously-administered radiation treatment plans from the database that are within the threshold difference from the predicted treatment plan;
identifying a set of treatment results, each of which is associated with one of the set of identified treatment plans;
receiving one or more criteria in a user interface, wherein the one or more criteria are features of a medical history;
filtering the set of identified treatment results by determining a similarity, within a threshold difference, between the patient who is to receive treatment and the other patients associated with the identified treatment results based in part on the one or more received criteria;
providing for display to a user in the user interface the one or more filtered treatment results for selection by the user as acceptable treatment results in the radiation treatment plan being created for the patient.

2. The method of claim 1, wherein the data further comprises one or more of the following: visual representations of an interior of the patient's body for medical purposes, a set of contours identifying a three-dimensional tumor volume and a plurality of anatomical structures located in a surrounding region of the tumor volumes, physics parameters, prescription parameters, and disease parameters.

3. The method of claim 1, wherein the one or more criteria comprise one or more of the following: available therapeutic options, a patient's ability to pay for available therapies, genetic results, digital genetics, in vitro lab results, prior treatment history, and comorbidities.

4. The method of claim 1, wherein a treatment result represents a set of one or more adverse events, wherein each adverse event is associated with one or more of the following outcomes: a probability of level of toxicity to each identified anatomical structure and a highest probable level of toxicity to each identified anatomical structure.

5. The method of claim 1, wherein a treatment result indicates one or more of the following parameters: a probability of tumor recurrence, a type of tumor recurrence that is either a local recurrence or a distant recurrence, and a probable length of time before tumor recurrence.

6. The method of claim 1, further comprising:
presenting, by the user interface, at least one parameter corresponding to the one or more filtered treatment results, the at least one parameter comprising one or more of the following: a level of toxicity to one or more anatomical structures near to a tumor, and an efficacy of the previously-administered treatment plan.

7. The method of claim 1, further comprising:
receiving, via the user interface, one or more user inputs for filtering or modifying the set of displayed treatment results.

8. A method for creating a radiation treatment plan for a given patient, the method comprising:
receiving data associated with a patient who is to receive radiation treatment, wherein the data comprises a medical history of the patient;
generating a plurality of features associated with the patient based on the received data;
providing the plurality of generated features to a prediction model to predict a treatment plan for the patient, wherein the treatment plan comprises a radiation treatment dose to be administered to the patient;
searching a database of previously-administered radiation treatment plans of other patients for treatment plans that are within a threshold difference from the predicted treatment plan, each previously-administered radiation treatment plan including a treatment result;
identifying a set of previously-administered radiation treatment plans from the database that are within the threshold difference from the predicted treatment plan;
identifying a set of treatment results, each of which is associated with one of the set of identified treatment plans;
providing for display to a user in a user interface the set of identified treatment results for selection by the user as acceptable treatment results in the radiation treatment plan being created for the patient;
receiving one or more criteria in the user interface regarding the display of the identified treatment results, wherein the one or more criteria are features of a medical history;
filtering the set of identified treatment results displayed on the user interface by determining a similarity, within a threshold difference, between the patient who is to receive treatment and the other patients associated with the identified treatment results based in part on the one or more received criteria; and
updating the user interface to display the set of filtered treatment results.

9. The method of claim 8, wherein the data further comprises one or more of the following: visual representations of an interior of the patient's body for medical purposes, a set of contours identifying a three-dimensional tumor volume and a plurality of anatomical structures located in a surrounding region of the tumor volumes, physics parameters, prescription parameters, and disease parameters.

10. The method of claim 8, wherein the one or more criteria comprise one or more of the following: available therapeutic options, a patient's ability to pay for available therapies, genetic results, digital genetics, in vitro lab results, prior treatment history, and comorbidities.

11. The method of claim 8, wherein a treatment result represents a set of one or more adverse events, wherein each adverse event is associated with one or more of the following outcomes: a probability of level of toxicity to each identified anatomical structure and a highest probable level of toxicity to each identified anatomical structure.

12. The method of claim 8, wherein a treatment result indicates one or more of the following: a probability of tumor recurrence, a type of tumor recurrence that is either a local recurrence or a distant recurrence, and a probable length of time before tumor recurrence.

13. The method of claim 8, further comprising:
presenting, by the user interface, at least one parameter corresponding to the one or more filtered treatment results, the at least one parameter comprising one or more of the following: a level of toxicity to one or more anatomical structures near to a tumor, and an efficacy of the previously-administered treatment plan.

14. A system for creating a radiation treatment plan for a given patient, the method comprising:
a database of previously-administered radiation treatment plans of other patients for treatment plans;
memory that stores computer-executable instructions; and
at least one processor configured to execute the computer-executable instructions, which, when executed, cause the at least one processor to perform operations comprising:
receiving data associated with a patient who is to receive radiation treatment, wherein the data comprises a medical history of the patient;
generating a plurality of features associated with the patient based on the received data;
providing the plurality of generated features to a prediction model to predict a treatment plan for the patient, wherein the treatment plan comprises a radiation treatment dose to be administered to the patient;
searching the database for treatment plans that are within a threshold difference to the predicted treatment plan, wherein each previously-administered radiation treatment plan is associated with a treatment result of the previously-administered radiation treatment plan;
identifying a set of previously-administered radiation treatment plans from the database that are within the threshold difference to the predicted treatment plan;
identifying a set of treatment results that are associated with the set of identified treatment plans;
receiving one or more criteria in a user interface, at least one of the one or more criteria comprising features of a medical history;
filtering the set of identified treatment results by determining a similarity, within a threshold difference, between the patient who is to receive radiation treatment and the other patients associated with the identified treatment results based in part on the one or more received criteria; and
providing for display to a user in the user interface the one or more filtered treatment results for selection by the user as acceptable treatment results in the radiation treatment plan being created for the patient.

15. The system of claim 14, wherein the data further comprises one or more of the following: visual representations of an interior of the patient's body for medical purposes, a set of contours identifying a three-dimensional tumor volume and a plurality of anatomical structures located in a surrounding region of the tumor volumes, physics parameters, prescription parameters, and disease parameters.

16. The system of claim 14, wherein the one or more criteria comprise one or more of the following: available therapeutic options, a patient's ability to pay for available therapies, genetic results, digital genetics, in vitro lab results, prior treatment history, and comorbidities.

17. The system of claim 14, wherein a treatment result represents a set of one or more adverse events, wherein each adverse event is associated with one or more of the following outcomes: a probability of level of toxicity to each identified anatomical structure and a highest probable level of toxicity to each identified anatomical structure.

18. The system of claim 14, wherein a treatment result indicates one or more of the following: a probability of tumor recurrence, a type of tumor recurrence that is either a local recurrence or a distant recurrence, and a probable length of time before tumor recurrence.

19. The system of claim 14, the computer-executable instructions further comprising:
presenting, by the user interface, at least one parameter corresponding to the one or more filtered treatment results.

20. The system of claim 14, the computer-executable instructions further comprising:
receiving, via the user interface, one or more user inputs for filtering or modifying the set of displayed treatment results.
